(19) United States
(12) Patent Application Publication
(10) Pub. No.: US 2004/0193155 A1
(43) Pub. Date: Sep. 30, 2004

(54) Fracture fixation plate with particular plate hole and fastener engagement and methods of using the same

(75) Inventor: Javier Castaneda, Miami, FL (US)

Correspondence Address: GORDON & JACOBSON, P.C., 65 WOODS END ROAD, STAMFORD, CT, 06905, US

(73) Assignee: Hand Innovations, Inc.

(21) Appl. No.: 10/724,305

(22) Filed: Nov. 29, 2003

Related U.S. Application Data
(63) Continuation-in-part of application No. 10/689,797, filed on Oct. 21, 2003.
Application No. 10/689,797 is a continuation-in-part of application No. 10/664,371, filed on Sep. 17, 2003.
Application No. 10/664,371 is a continuation-in-part of application No. 10/401,089, filed on Mar. 27, 2003.

(51) Int. Cl. A61B 17/58

(52) U.S. Cl. 606/60

(57) ABSTRACT

A bone fracture fixation system includes a substantially rigid plate defining a first hole having at least one first thread at a first depth, and a second hole, a first fastener having a head portion and a shaft portion, with the head portion having a second thread with a second depth substantially greater than said first depth, and a second fastener sized for use within the second hole. In addition, a bone fracture fixation system includes a rigid plate defining at least one cylindrical first hole having at least two discrete helical threads each with a entry lead offset by a predetermined angular displacement, a fastener having a head portion with a thread sized for engagement within the first hole and a shaft portion, wherein when the first fastener is engaged in the first hole, the thread of the head engages with only one of the discrete helical threads.

This application is a continuation-in-part of U.S. Ser. No. 10/689,797, filed Oct. 21, 2003, which is a continuation-in-part of U.S. Ser. No. 10/664,371, filed Sep. 17, 2003, which is a continuation-in-part of U.S. Ser. No. 10/401,089, filed Mar. 27, 2003, all of which are hereby incorporated by reference herein in their entireties.

### BACKGROUND OF THE INVENTION

1. Field of the Invention

This invention relates broadly to surgical implants. More particularly, this invention relates to a bone fracture fixation system for distal radius fractures.

2. State of the Art

Fracture to the metaphyseal portion of a long bone can be difficult to treat. Improper treatment can result in deformity and long-term discomfort.

By way of example, a Colles' fracture is a fracture resulting from compressive forces being placed on the distal radius, and which causes backward or dorsal displacement of the distal fragment and radial deviation of the hand at the wrist. Often, a Colles' fracture will result in multiple bone fragments which are movable and out of alignment relative to each other. If not properly treated, such fractures may result in permanent wrist deformity and limited articulation of the wrist. It is therefore important to align the fracture and fixate the bones relative to each other so that proper healing may occur.

Alignment and fixation of a metaphyseal fracture (occurring at the extremity of a shaft of a long bone) are typically performed by one of several methods: casting, external fixation, interosseous wiring, and plating. Casting is non-invasive, but may not be able to maintain alignment of the fracture where many bone fragments exist. Therefore, as an alternative, external fixators may be used. External fixators utilize a method known as ligamentotaxis, which provides distraction forces across the joint and permits the fracture to be aligned based upon the tension placed on the surrounding ligaments. However, while external fixators can maintain the position of the wrist bones, it may nevertheless be difficult in certain fractures to first provide the bones in proper alignment. In addition, external fixators are often not suitable for fractures resulting in multiple bone fragments. Interosseous wiring is an invasive procedure whereby screws are positioned into the various fragments and the screws are then wired together as bracing. This is a difficult and time-consuming procedure. Moreover, unless the bracing is quite complex, the fracture may not be properly stabilized. Plating utilizes a stabilizing metal plate typically against the dorsal side of the bones, and a set of parallel pins extending from the plate into holes drilled in the bone fragments to provide stabilized fixation of the fragments. However, many currently available plate systems fail to provide desirable alignment and stabilization.

In particular, with a distal radius fracture the complex shape of the distal radius, including the bulky volar rim of the lunate fossa, relatively flat volar rim of the scaphoid fossa, and volar marginal fragment from the lunate fossa should be accommodated. A fixation plate should provide desirable alignment and stabilization of both the subchondral bone and the articular surfaces of the distal radius.

### SUMMARY OF THE INVENTION

It is therefore an object of the invention to provide an improved fixation system for distal radius fractures.

It is another object of the invention to provide a distal radius volar fixation system that desirably aligns and stabilizes multiple bone fragments in a fracture to permit proper healing.

It is also an object of the invention to provide a distal radius volar plate system which provides support for articular and subchondral surfaces.

It is an additional object of the invention to provide a distal radius volar plate system which accommodates the anatomical structure of the metaphysis of the distal radius.

It is a further object of the invention to provide a distal radius volar plate system which provides support without interfering with ligaments and soft tissues near the edge of the articular surface.

In accord with these and other objects, which will be discussed in detail below, a distal radius volar fixation system is provided. The system generally includes a plate intended to be positioned against the volar side of the radius, a plurality of bone screws for securing the plate along a non-fractured portion of the radius bone, a plurality of bone pegs sized to extend from the plate and into bone fragments at the metaphysis of a radius bone, and one or more K-wires to facilitate alignment and fixation of the plate over the bone and guide the process of application. Preferred bone pegs and peg holes within the plate are provided which facilitate entry and retention of the bone pegs within the peg holes.

The plate is generally T-shaped, defining an elongate body and a generally transverse head angled upward relative to the body, and includes a first side which is intended to contact the bone, and a second side opposite the first side. The body includes a plurality of countersunk screw holes for the extension of the bone screws therethrough, and optionally one or more substantially smaller alignment holes. The lower surfaces of the radial and ulnar side portions of the head are contoured upward (in a Z direction) relative to the remainder of the head to accommodate the lunate and scaphoid processes. An extension is provided at the head portion along the distal ulnar side of the head to buttress the volar lip (marginal fragment) of the lunate fossa of the radius bone, thereby providing support to maintain the wrist within the articular socket. Moreover, the contoured shape provides a stable shape that prevents rocking of the plate on the bone. The upper and lower surfaces are chamfered to have a reduced profile that limits potential interface with the ligaments and soft tissue near the edge of the lunate fossa. The head includes a plurality of threaded peg holes for receiving the pegs therethrough. The peg holes are arranged into a first set provided in a proximal portion of the head, and a second relatively distal set preferably provided in the tapered portion of the head.

The first set of the peg holes is substantially linearly arranged generally laterally across the head. The line of pegs is preferably slightly oblique relative to a longitudinal axis through the body of the plate. Axes through the first set of holes are preferably oblique relative to each other, and are preferably angled relative to each other in two dimensions such that pegs inserted therethrough are similarly obliquely angled relative to each other. The pegs in the first set of peg holes provide support for the dorsal aspect of the subchondral bone fragments.

The second set of peg holes is provided relatively distal of the first set. The holes of the second set, if more than one are provided, are slightly out of alignment but generally linearly arranged. The pegs in the second set of peg holes provide support for the volar aspect of the subchondral bone, behind and substantially parallel to the articular bone surface.

A distal alignment hole is provided generally between two peg holes of the second set of peg holes. At the upper surface of the plate, the distal alignment hole is substantially cylindrical, while at the lower surface, the hole is laterally oblong. One or more proximal alignment holes of a size substantially smaller than the peg holes are provided substantially along a distal edge defined by a tangent line to shafts of pegs inserted in the first set of peg holes, and facilitate temporary fixation of the plate to the bone with K-wires. Furthermore, along the body two longitudinally displaced alignment holes are also provided. All of the alignment holes are sized to closely receive individual K-wires.

The plate may be used in at least two different manners. According to a first use, the surgeon reduces a fracture and aligns the plate thereover. The surgeon then drills K-wires through the proximal alignment holes to temporarily fix the orientation of the head of the plate to the distal fragment. Once the alignment is so fixed, the fracture is examined, e.g., under fluoroscopy, to determine whether the K-wires are properly aligned relative to the articular surface. As the axes of the proximal alignment holes correspond to axes of adjacent peg holes, the fluoroscopically viewed K-wires provide an indication as to whether the pegs will be properly oriented. If the placement is correct, the K-wires maintain the position of the plate over the fracture. The peg holes may then be drilled with confidence that their locations and orientations are proper. If placement is not optimal, the K-wires can be removed and the surgeon has an opportunity to relocate and/or reorient the K-wires and drill again. Since each K-wire is of relatively small diameter, the bone is not significantly damaged by the drilling process and the surgeon is not committed to the initial drill location and/or orientation.

According to a second use, the plate may be used to correct a metaphyseal deformity (such as malformed fracture or congenital deformity). For such purposes, a K-wire is drilled into the bone parallel to the articular surface in the lateral view under fluoroscopy until one end of the K-wire is located within or through the bone and the other end is free. The free end of the K-wire is guided through the distal oblong alignment hole of the head of the plate, and the plate is slid down over the K-wire into position against the bone. The oblong alignment hole permits the plate to tilt laterally over the K-wire to sit flat on the bone, but does not permit movement of the plate over the K-wire in the anterior-posterior plane. The surgeon drills holes in the bone in alignment with the peg holes and then fixes the plate relative the bone with pegs. The bone is then cut, and the body of the plate is levered toward the shaft of the bone to re-orient the bone. The body of the plate is then fixed to the shaft to correct the anatomical defect.

Additional objects and advantages of the invention will become apparent to those skilled in the art upon reference to the detailed description taken in conjunction with the provided figures.

### BRIEF DESCRIPTION OF THE DRAWINGS

FIG. 1 is a radial side elevation of a right-hand volar plate according to the invention, shown with pegs coupled thereto;

FIG. 2 is an ulnar side elevation of a right-hand volar plate according to the invention, shown with pegs coupled thereto;

FIG. 3 is top view of a right-hand volar plate according to the invention, shown with pegs and screws;

FIG. 4 is bottom view of a right-hand volar plate according to the invention, shown with pegs coupled thereto;

FIG. 5 is a perspective view of a right-hand volar plate according to the invention, shown with pegs coupled thereto and K-wires extending through body and proximal head alignment holes;

FIG. 6 is a front end view of a right-hand volar plate according to the invention, shown with pegs coupled thereto and K-wires extending through alignment holes;

FIGS. 7 through 12 illustrate a method of performing an osteotomy of the distal radius according to the invention;

FIG. 13 is a side elevation of a partially threaded peg according to the invention; and

FIG. 14 is a schematic illustration of a peg coupled within a peg hole according to one embodiment of the invention.

### DETAILED DESCRIPTION OF THE PREFERRED EMBODIMENTS

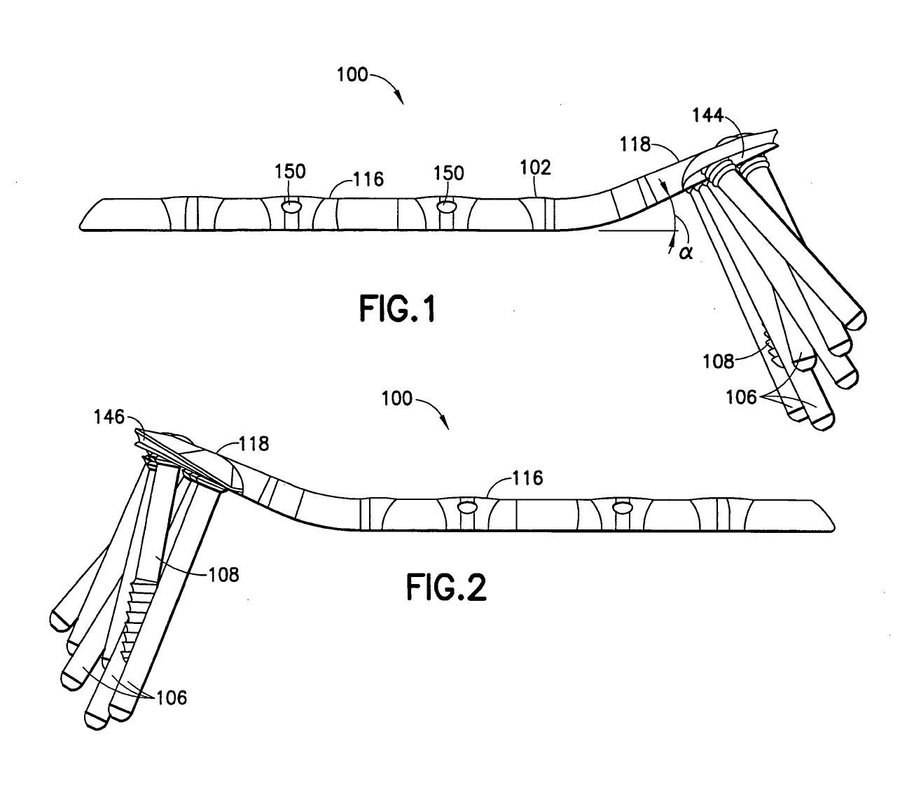

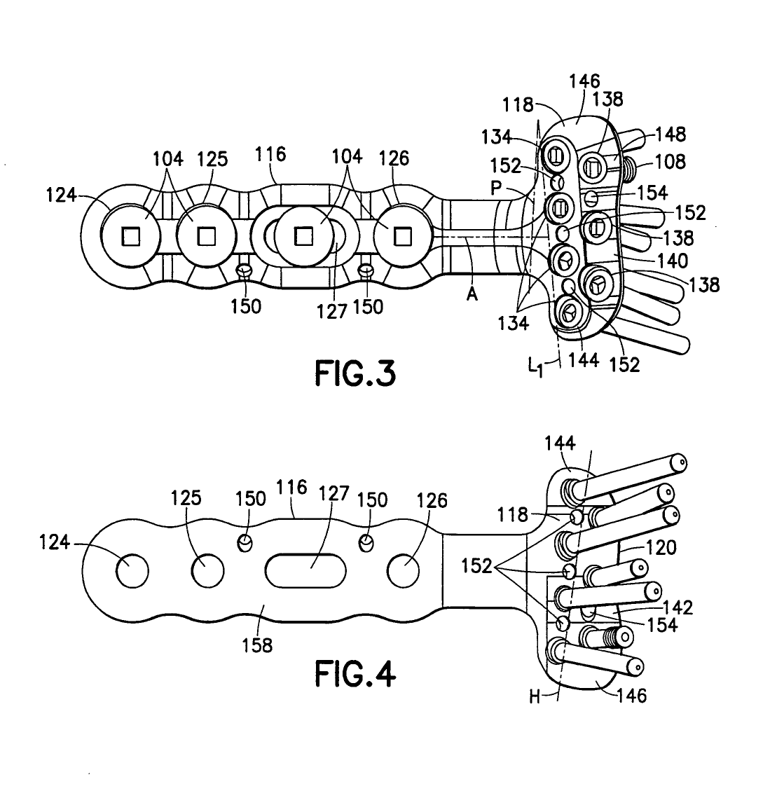

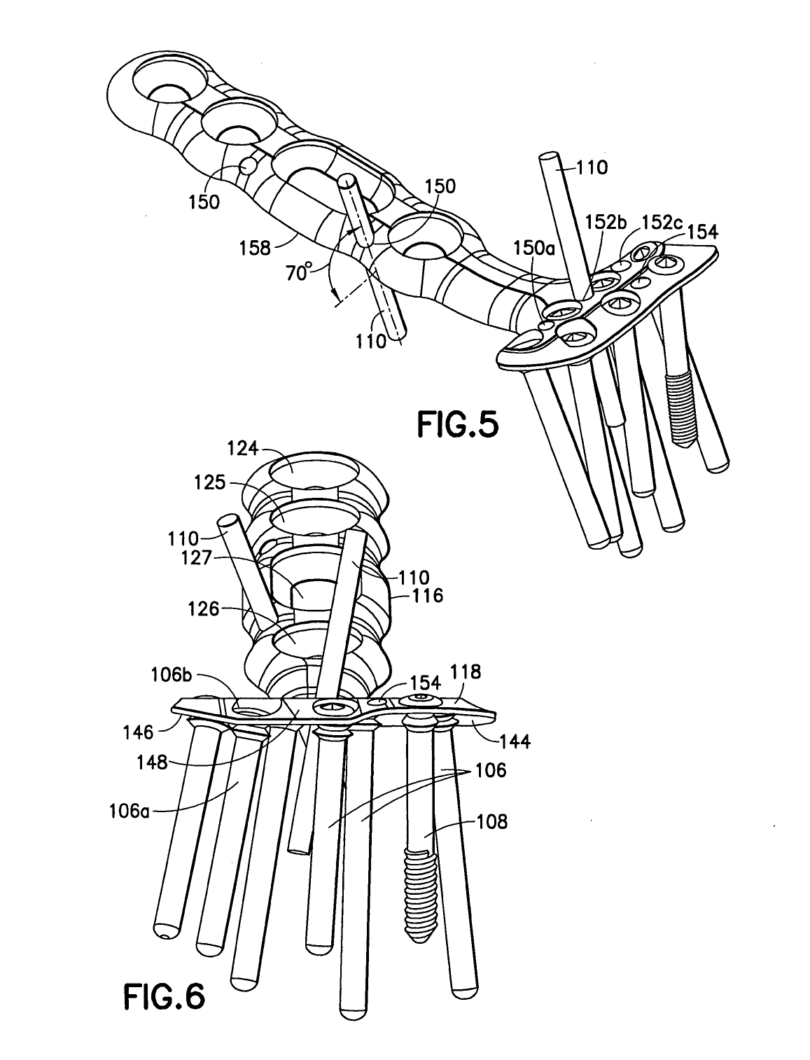

Turning now to FIGS. 1 through 6, a fracture fixation system 100 according to the invention is shown. The system 100 is particularly adapted for aligning and stabilizing multiple bone fragments in a dorsally displaced distal radius fracture (or Colles' fracture). The system 100 generally includes a substantially rigid T-shaped plate 102, commonly called a volar plate, bone screws 104 (FIG. 3), pegs 106, 108, and K-wires 110 (FIGS. 5 and 6). Pegs 106 have a threaded head and a non-threaded shaft, and pegs 108 have both a threaded head and a threaded shaft. Either pegs 106 or 108, or a combination thereof may be used at the discretion of the surgeon. Exemplar pegs are described in more detail in U.S. Pat. No. 6,364,882, which is hereby incorporated by reference herein in its entirety.

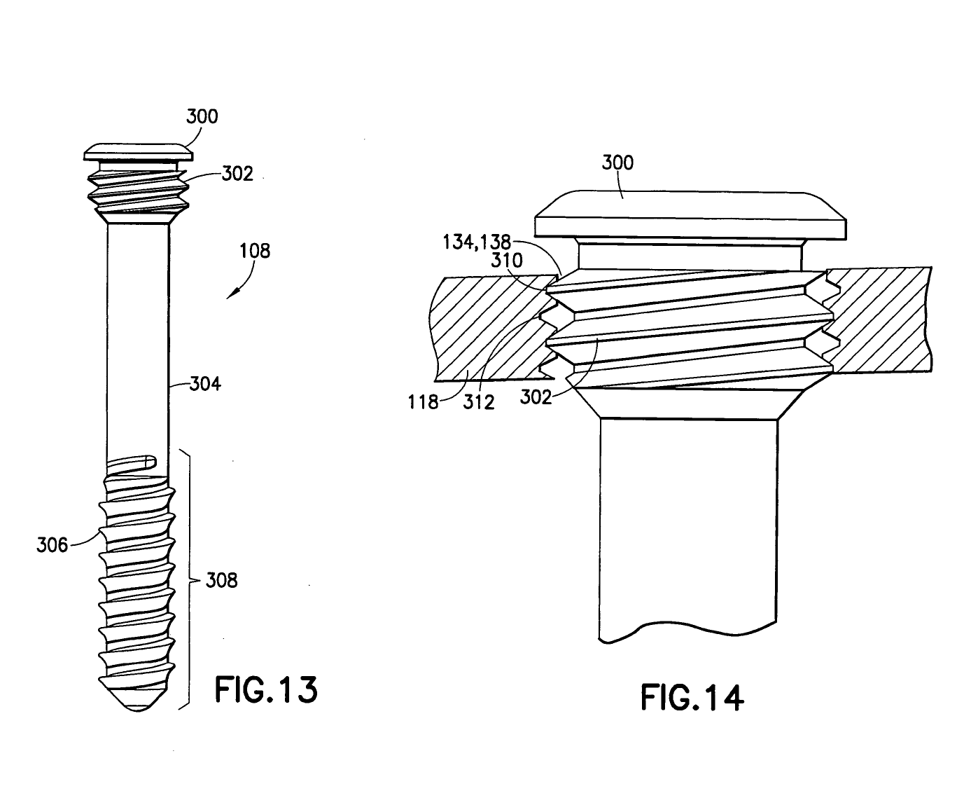

In addition, a preferred partially threaded shaft peg 108 is shown best in FIGS. 6 and 13. Peg 108 includes a head portion 200 with preferably a single helical machine thread 202 of a first pitch and a shaft 204 portion having one or more threads 206 of a larger second pitch. (The head portion of non-threaded shaft pegs 106 also preferably includes a single helical thread.) The threads 206 preferably extend along a distal portion 208 of the shaft 204, and most preferably where such distal portion comprises approximately one-half the length of the shaft. Alternatively, or in addition, one or more pegs may be used where the threads extend along substantially the entirety, or the entirety, or the length of the shaft.

The volar plate 102 shown in the figures is a right-hand plate intended to be positioned against the volar side of a fractured radius bone of the right arm. It is appreciated that a left-hand volar plate is substantially a mirror image of the plate shown and now described. The T-shaped plate 102 defines an elongate body 116, and a head 118 angled upward (in the Z-direction) relative to the head. The angle a between the head 118 and the body 116 is preferably approximately 25°. The head 118 includes a distal buttress 120 (i.e., the portion of the head distal a first set of peg holes 134, discussed below). The plate 102 has a thickness of preferably approximately 0.1 inch, and is preferably made from a titanium alloy, such as Ti-6A1-4V.

Referring to FIG. 4, the body 116 includes four preferably countersunk screw holes 124, 125, 126, 127 for the extension of bone screws 104 therethrough (FIG. 2). One of the screw holes, 127, is preferably generally oval in shape permitting longitudinal movement of the plate 102 relative to the shaft of a bone screw when the screw is not tightly clamped against the plate.

Referring to FIGS. 3 and 4, according to one preferred aspect of the plate 102, the head portion 118 includes a first set of threaded preferably cylindrical peg holes 134 (for placement of pegs 106 and/or 108 therein) and a second set of threaded preferably cylindrical peg holes 138 (for placement of pegs 106 and/or 108 therein). Referring to FIG. 14, the peg holes 134, 138 optionally have double lead internal threads 210, 212, with entries to these threads located 180° apart. Each of the threads 210, 212 is adapted to mate securely with the thread 202 on a peg head 200, however thread 202 can only mate with one of the threads 210, 212 at any one time. The depth of each of the double lead internal threads 210, 212 is preferably substantially less than the depth of thread 202 on peg head 200, and most preferably approximately one half such depth. The double lead threads 210, 212 facilitate alignment and entry of the peg head thread 202 into a thread of the peg hole, as the peg will require rotation by at most 180° in a single rotational direction before thread engagement. Furthermore, in distinction from a conical head and hole, the cylindrical double lead thread hole does not compromise the secure interlock attained from full travel of the thread 202 of the peg head 200 through the cylindrical peg hole 134, 138 through, e.g., 900°. Moreover, the double lead threads reduce cross-threading by fifty percent, whether a single lead thread or a double-lead thread peg is used.

Referring back to FIGS. 3 and 4, the peg holes 134 of the first set are arranged substantially parallel to a line L₁ that is preferably slightly skewed (e.g., by 5°−10°) relative to a perpendicular P to the axis A of the body portion 116. Axes through the first set of peg holes (indicated by the pegs 106 extending therethrough) are preferably oblique relative to each other, and are preferably angled relative to each other in two dimensions, generally as described in commonly-owned U.S. Pat. No. 6,364,882, which is hereby incorporated by reference herein in its entirety. This orientation of the pegs operates to stabilize and secure the head 118 of the plate 102 on the bone even where such pegs 106 do not have threaded shafts.

The second set of peg holes 138 is provided relatively distal of the first set of peg holes 134 and is most preferably primarily located in the buttress 120. Each of the peg holes 138 preferably defines an axis that is oblique relative to the other of peg holes 136 and 138. Thus, each and every peg 106, 108 when positioned within respective peg holes 134, 138 defines a distinct axis relative to the other pegs. Moreover, the axes of the peg holes 138 are preferably oriented relative to the axes of peg holes 134 such that pegs 106, 108 within peg holes 138 extend (or define axes which extend) between pegs (or axes thereof) within peg holes 134 in an interleaved manner.

Referring specifically to FIGS. 1, 2, 5 and 6, according to another preferred aspect of the plate 102, in order to approximate the anatomy for ideal fracture support and maintain a low profile, the upper and lower surfaces 140, 142, respectively of the buttress 120 are chamfered, with the chamfer of the lower surface 142 being contoured for the anatomical structure that it will overlie. In particular, the lower surface 142 at an ulnar-side portion 144 of the head portion 118 is elevated primarily in a distal direction to accommodate the bulky volar rim of the lunate fossa, and the lower surface 142 at a radial side portion 146 of the head 118 is elevated laterally relative to the remainder of the head to accommodate a prominence at the radial aspect of the bone, as indicated by the visibility of these lower surfaces in the side views of FIGS. 1 and 2 and head-on view of FIG. 6. The contoured shape (with generally three defined planes) provides a stable shape that prevents rocking of the plate on the bone. In addition, the upper and lower surfaces 140, 142 are chamfered to have a reduced profile that limits potential interface with the ligaments and soft tissue (e.g., tendons) near the edge of the articular surface. A distal extension 148 is also provided at the ulnar side portion 146 to further buttress the volar lip (volar marginal fragment of the lunate fossa) of the articular socket of the radius bone, thereby providing support to maintain the wrist within the articular socket.

Referring specifically to FIGS. 3 and 4, according to a further preferred aspect of the invention, the plate 102 is provided with body alignment holes 150, proximal head alignment holes 152a, 152b, 152c (generally 152), and a distal head alignment hole 154, each sized to closely accept standard Kirschner wires (K-wires), e.g., 0.7-1.2 mm in diameter. The upper openings of all the alignment holes 150, 152, 154 are substantially smaller in diameter (e.g., by thirty to fifty percent) than the shafts of screws 104 (approximately 3.15 mm in diameter) and the shafts of pegs 106, 108 (approximately 2.25 mm in diameter). The body alignment holes 150 are longitudinally displaced along the body portion 116 and provided at an oblique angle (preferably approximately 70°, as shown in FIG. 5) relative to the lower surface 158 of the body portion 116. The proximal head alignment holes 152 alternate with the peg holes 134. A tangent line H to the distalmost points of the head alignment holes 152 is preferably substantially coincident or closely parallel with a line tangent to points on the circumferences of the shafts of pegs 106 inserted through holes 134 adjacent the head portion 118 of the plate 102. With respect to the proximal head alignment holes, it is appreciated that a shaft 106a of a peg is generally smaller in diameter than a head 106b of a peg (FIG. 6). Thus, a line tangent to the peg holes 134 (each sized for receiving the head 106b of peg 106) will be closely located, but parallel, to a line tangent to a distalmost point on the respective alignment hole 152. Nevertheless, for purposes of the claims, both (i) a tangent line which is preferably substantially coincident with a line tangent to points on the circumferences of the shafts of pegs and (ii) a tangent line to a set of peg holes shall be considered to be “substantially coincident” with a line tangent to a distalmost point of an alignment hole 152. Axes through alignment holes 152 preferably generally approximate (within, e.g., 3°) the angle of an axis of an adjacent peg hole 134. Moreover, the axis through each proximal alignment hole 152 is preferably oriented substantially equidistantly between the axes through peg holes 134 on either side of the alignment hole. As such, K-wires 110 inserted into the proximal alignment holes 152 (and extending coaxial with the axes therethrough) define a virtual surface which is substantially the same virtual surface defined by pegs 106, 108 inserted through peg holes 134. This common virtual surface follows the dorsal aspect of the subchondral bone. Thus, as described in more detail below, the insertion of K-wires 110 through proximal alignment holes 152 provides a visual cue to the surgeon regarding the alignment of the plate 102 and subsequently inserted pegs 106, 108. Distal head alignment hole 154 is provided between the central and radial-side peg holes 138, and has a circular upper opening, and a laterally oblong lower opening, as shown best in FIG. 6.

The plate may be used in at least two different applications: fracture fixation and correction of a metaphyseal deformity. In either application, an incision is first made over the distal radius, and the pronator quadratus is reflected from its radial insertion exposing the entire distal radius ulnarly to the distal radioulnar joint. For fracture fixation, the surgeon reduces the fracture and aligns the plate 102 thereover. The surgeon then drills preferably two K-wires 110 through respective body alignment holes 150, and preferably a plurality of K-wires through selected proximal head alignment holes 152 at the location at which the surgeon believes the pegs 106, 108 should be placed based on anatomical landmarks and/or fluoroscopic guidance. The K-wires temporarily fix the orientation of the plate to the distal fragment. While the fixation is temporary, it is relatively secure in view of the fact that the body alignment holes 150, proximal head alignment holes 152, and K-wires 110 therethrough are angled in different orientations relative to the lower surface of the plate. Once the alignment is so fixed, the fracture is examined, e.g., under fluoroscopy, to determine whether the K-wires 110 are properly aligned relative to the articular surface. As the axes of the proximal head alignment holes 152 correspond to axes of the adjacent peg holes 134, the fluoroscopically viewed K-wires 110 provide an indication as to whether the pegs 106, 108 will be properly oriented. If the placement is correct, the K-wires 110 maintain the position of the plate 102 over the fracture while holes in the bone are drilled through the screw holes 124, 125, 126, 127 for the screws 104 and peg holes 134, 138 for pegs 106, 108, with confidence that the locations and orientation of the screws and pegs inserted therein are anatomically appropriate. In addition, where pegs 108 are used, due to the difference in pitch between the head threads 202 and shaft threads 206, slight compression of a distally or dorsally displaced fragment toward a proximal fragment or bone (e.g., 1.5 mm of travel) is effected even though the head 200 will lock relative to the head 118 of the plate 100. Once the screws 104 and pegs 106, 108 have secured the plate to the bone, the K-wires are preferably removed.

If fluoroscopic examination indicates that placement of the K-wires 110 is not optimal, the K-wires can be removed and the surgeon has an opportunity to relocate and/or reorient the K-wires and drill again. Since each K-wire is of relatively small diameter, the bone is not significantly damaged by the drilling process and the surgeon is not committed to the initial drill location and/or orientation.

The pegs 106 within peg holes 138 define projections that provide support at the volar aspect behind the articular surface of the bone surface. The sets of pegs 106, 108 through peg holes 134, 138 laterally overlap so that the pegs preferably laterally alternate to provide closely-spaced tangential cradling of the subchondral bone. A preferred degree of subchondral support is provided with four peg holes 134 (and associated pegs) through the proximal portion of the head 118 of the plate, and three peg holes 138 (and associated pegs) through the distal portion of the head 118. The fracture fixation system thereby defines a framework which substantially tangentially supports the bone fragments in their proper orientation. In accord with an alternate less preferred embodiment, suitable support may also be provided where the pegs 106 and 108 are parallel to each other or in another relative orientation or with fewer peg holes and/or pegs.

The method particularly facilitates stabilization of a metaphyseal fracture which may include a smaller distal bone fragment spaced apart from a larger proximal fragment. The insertion of one or more threaded pegs 108 (preferably in conjunction with several non-threaded pegs 106) in which the threads on the shaft 206 have a pitch greater than the threads 202 on the head 200 causes a limited amount of compression of the smaller distal bone fragment toward the larger proximal bone fragment, and thus toward the plate.

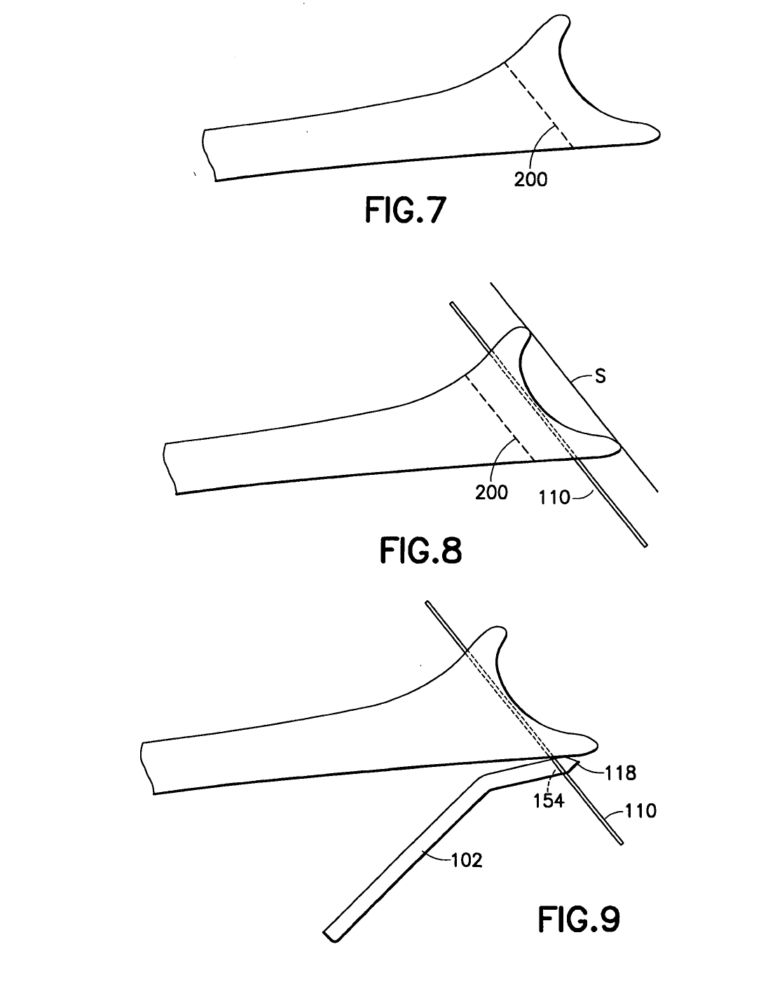

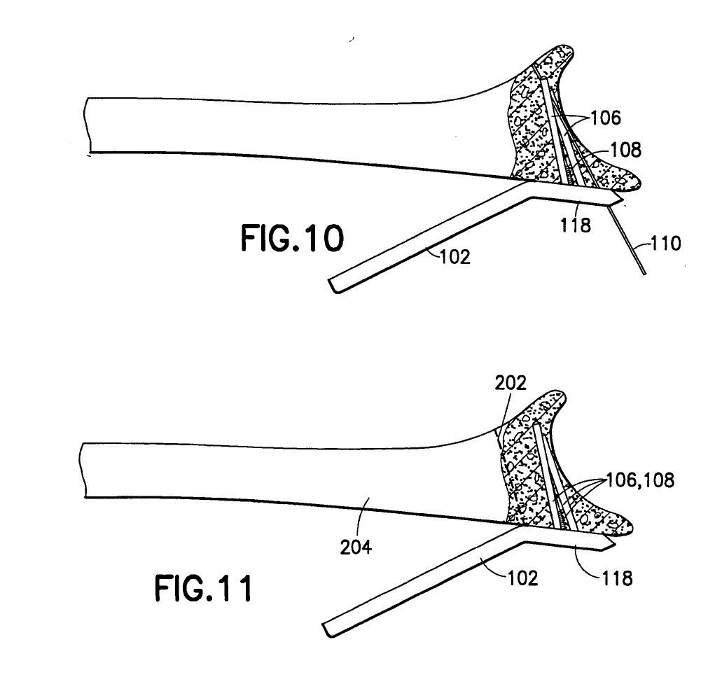

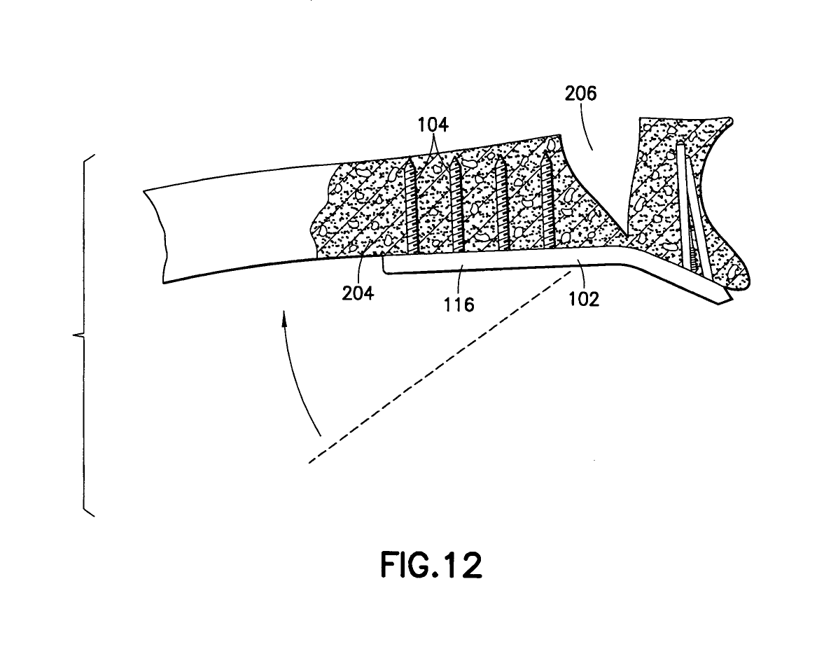

According to a second use, the plate may be used to correct a metaphyseal deformity 200 (such as malformed fracture or congenital deformity), as shown in FIG. 7. For such purposes, a K-wire 110 is drilled into the bone parallel to the articular surface S in the lateral view under fluoroscopy (FIG. 8). The free end of the K-wire 110 is guided through the oblong distal head alignment hole 154, and the plate 102 is slid down over the K-wire into position against the bone (FIG. 9). The oblong alignment hole 154 permits the plate 102 to tilt laterally over the K-wire 110 to sit flat on the bone, but does not permit tilting of plate relative to the K-wire in the anterior-posterior plane. Once the plate 102 is seated against the bone, the surgeon drills holes in the bone in alignment with the peg holes 134, 138 (FIG. 3) and then fixes the plate relative the bone with pegs 106, 108 (FIG. 10). The K-wire 110 is removed. The bone is then saw cut at 202 proximal the location of the head 118 of the plate 102 (FIG. 11), and the body 116 of the plate is levered toward the proximal diaphyseal bone 204, creating an open wedge 206 at the deformity (FIG. 12). When the body 116 of the plate 102 is in contact and longitudinal alignment with the diaphysis of the bone, the bone distal of the cut has been repositioned into the anatomically correct orientation relative to the shaft of the bone. The body 116 of the plate 102 is then secured to the bone with screws 104. Post-operatively, the open wedge in the bone heals resulting in an anatomically correct distal radius.

While fixed single-angle pegs have been disclosed for use with the plate (i.e., the pegs may be fixed in respective threaded peg holes 134, 136 only coaxial with an axis defined by the respective peg holes), it is appreciated that an articulating peg system, such as that disclosed in co-owned U.S. Pat. No. 6,440,135 or co-owned and co-pending U.S. Ser. No. 10/159,612, both of which are hereby incorporated by reference herein in their entireties, may also be used. In such articulating peg systems, the peg holes and pegs are structurally adapted such that individual pegs may be fixed at any angle within a range of angles. In addition, while less preferable, one or both sets of the pegs may be replaced by preferably blunt tines which are integrated into the plate such that the plate and tines are unitary in construct. Similarly, other elongate projections may be coupled to the plate to define the desired support.

There have been described and illustrated herein embodiments of a fixation plate, and particularly plates for fixation of distal radius fractures, as well as a method of aligning and stabilizing a distal radius fracture and performing an osteotomy. While particular embodiments of the invention have been described, it is not intended that the invention be limited thereto, as it is intended that the invention be as broad in scope as the art will allow and that the specification be read likewise. Thus, while particular materials, dimensions, and relative angles for particular elements of the system have been disclosed, it will be appreciated that other materials, dimensions, and relative angles may be used as well. In addition, while a particular number of screw holes in the volar plate and bone screws have been described, it will be understood another number of screw holes and screws may be provided. Further, fewer screws than the number of screw holes may be used to secure to the plate to the bone. Also, fewer or more peg holes and bone pegs may be used, preferably such that at least two pegs angled in two dimensions relative to each other are provided. In addition, while a particular preferred angle between the head and body has been disclosed, other angles can also be used. Moreover, while the cylindrical double lead thread hole and single thread head interface has been disclosed with respect to a fracture plate for distal radius fractures, it is appreciated that such a system has advantage to other orthopedic stabilization devices such as fragment plates (which may be rectangular in shape or a different shape) and plates specifically designed for fractures of other bones. Similarly, a threaded peg (i.e., locking screw) with threads of different pitches on the head and along the shaft may also be used in other applications. Furthermore, while a double lead thread hole is preferred for use with a peg having a single thread on its head, it is appreciated that, e.g., a triple lead thread hole can be used where the entry leads are angularly offset by 120°. Such will reduce cross threading by two-thirds, but will also reduce hole thread depth further. Also, while the double lead thread system is described with respect to a bone plate, it is appreciated that it can be applied to other orthopedic implants, such as rods, nails, prostheses, etc., having holes for fixation. Furthermore, while the double lead thread hole has been shown in conjunction with a peg having a single lead thread on its head portion, it is appreciated that the double lead thread hole is perfectly adapted for use with a peg having a double lead thread on its head portion. It will therefore be appreciated by those skilled in the art that yet other modifications could be made to the provided invention without deviating from its spirit and scope.

What is claimed is:

1. An orthopedic system, comprising:
a) an orthopedic implant defining at least one first hole having at least one first thread at a first depth, and at least one second hole;
b) a first fastener having a head portion and a shaft portion, said head portion having a second thread with a second depth substantially greater than said first depth; and
c) a second fastener sized for use within said at least one second hole.

2. An orthopedic system according to claim 1, wherein:
said at least one first thread is two threads offset by approximately 180°

3. An orthopedic fixation system according to claim 2, wherein:
said second depth is approximately twice said first depth.

4. An orthopedic fixation system according to claim 2, wherein:
said at least one first hole is cylindrical.

5. An orthopedic fixation system according to claim 1, wherein:
said at least one first hole is cylindrical.

6. An orthopedic fixation system according to claim 1, wherein:
said shaft of said first fastener includes a third thread along at least a portion thereof.

7. An orthopedic fixation system according to claim 6, wherein:
said second and third threads have different pitches.

8. An orthopedic fixation system according to claim 7, wherein:
said pitch of said third thread is greater than said pitch of said second thread.

9. An orthopedic fixation system according to claim 6, wherein:
said third thread is provided along a distal portion of said shaft and is absent from a proximal portion of said shaft.

10. An orthopedic fixation system according to claim 1, wherein:
said implant includes a head portion configured and dimensioned to conform to a metaphysis of a bone and a shaft portion configured and dimensioned to conform to a diaphysis of a bone,
wherein said at least one first hole is provided in said head portion and said at least one second hole is provided in said shaft portion.

11. An orthopedic system according to claim 10, wherein:
said second fastener is a cortical screw sized to provide compression of said shaft portion of said implant against the bone.

12. An orthopedic system, comprising:
a) an orthopedic implant defining at least one substantially cylindrical first hole having at least two discrete helical threads each with an entry offset by a predetermined angular displacement; and
b) a first fastener having a head portion sized for engagement within said first hole and a shaft portion, said head portion having a thread,
wherein when said first fastener is engaged in said first hole, said thread of said head engages with only one of said discrete helical threads.

13. An orthopedic system according to claim 12, wherein:
said first hole includes exactly two helical threads with respective entries offset by approximately 180°.

14. An orthopedic system according to claim 13, wherein:
each of said two helical threads of the first hole has a depth of no more than one half the depth of the thread on the head portion of the first fastener.

15. An orthopedic system according to claim 12, wherein:
said shaft of said first fastener includes a thread along at least a portion thereof.

16. An orthopedic system according to claim 15, wherein:
said threads along said head and shaft portions are of different pitches.

17. An orthopedic system according to claim 16, wherein:
said pitch of said thread about said shaft portion is greater than said pitch of said thread about said head.

18. An orthopedic system according to claim 15, wherein:
said thread on said shaft is provided along a distal portion of said shaft and is absent from a proximal portion of said shaft.

19. An orthopedic system according to claim 12, wherein:
said implant includes a head portion configured and dimensioned to conform to a metaphysis of a bone and a shaft portion configured and dimensioned to conform to a diaphysis of a bone,
wherein said at least one first hole is provided in said head portion, and a second hole is provided in said shaft portion.

20. An orthopedic system according to claim 19, further comprising:
c) a cortical screw sized for use within said second hole to provide compression of said shaft portion of said plate against the bone.

21. A bone fixation system, comprising:
a) a plate including at least one threaded hole; and
b) a bone fastener including a head portion having a first machine thread with a first pitch, and a shaft portion having a second thread with a second pitch greater than said first pitch, said thread on said shaft is provided along a distal portion of said shaft and is absent from a proximal portion of said shaft,
wherein said head portion of said bone fastener is configured to threadedly engage with said at least one threaded hole.

22. A bone fixation system according to claim 21, wherein:
said threaded and non-threaded portions of said shaft of said fastener are substantially equal in length.

23. A bone fastener, comprising:
a) a head portion having a first machine thread with a first pitch; and
b) a shaft portion having a second thread with a second pitch greater than said first pitch, wherein said thread on said shaft is provided along a distal portion of said shaft and is absent from a proximal portion of said shaft.

24. A bone fastener according to claim 23, wherein:
said threaded and non-threaded portions of said shaft are substantially equal in length.

25. A method of stabilizing a metaphyseal fracture of long bone, comprising:
a) providing an orthopedic implant with a plate portion provided with a first hole having at least two angularly offset helical threads;
b) providing a first fastener having a head and a shaft, where the head has a single helical thread; and
c) inserting the first fastener through the first hole and into the bone and locking the head of the first fastener in only one of the angularly offset helical threads of the hole.

26. A method according to claim 25, further comprising prior to c), reducing the fracture.

27. A method according to claim 25, wherein:
the fracture includes a bone fragment spaced apart from the plate by a larger section of bone, and the shaft of the first fastener includes threads, such that said inserting the first fastener causes the threads on the first fastener to engage the bone fragment.

28. A method according to claim 27, wherein:
the threads on the shaft of the first fastener have a pitch greater than the threads on the head of the first fastener, such that said inserting the first fastener causes the threads on the first fastener to provide limited compression of the bone fragment against the larger section of bone in the direction of the plate.

29. A method according to claim 25, further comprising:
compressing the plate against the bone.

30. A method according to claim 25, wherein:
said providing an orthopedic implant includes providing a plate with a head portion configured and dimensioned to conform to a metaphysis of a bone and a shaft portion configured and dimensioned to conform to a diaphysis of a bone.

31. A method of stabilizing a metaphyseal fracture of long bone, wherein the metaphyseal fracture includes a smaller bone fragment spaced apart from a larger metaphyseal fragment of the bone, the method comprising:
a) providing an implant with a plate portion with a first hole having at least one helical thread;
b) providing a first fastener having a head and a shaft, where the head has a first helical thread at a first pitch for engagement with one of the at least one helical threads of the first hole, and the shaft has a distal portion with a second helical thread at a second pitch different than the first pitch, and a proximal portion from which the second helical thread is absent; and
c) inserting the first fastener through the first hole and into the larger and smaller bone fragments and locking the head of the first fastener relative to the first hole, wherein the rotational travel of the head of the first fastener through the first hole is sufficient to cause a limited amount of compression of the smaller fragment against the larger fragment.

32. A method of stabilizing a metaphyseal fracture of long bone, wherein the metaphyseal fracture includes a distal bone fragment spaced apart from a proximal bone fragment, the method comprising:
a) providing an implant with a plate portion with a first hole having at least one helical thread;
b) providing a first fastener having a head and a shaft, where the head has a first helical thread at a first pitch for engagement with one of the at least one helical threads of the first hole, and the shaft has a distal portion with a second helical thread at a second pitch different than the first pitch, and a proximal portion from which the second helical thread is absent; and
c) inserting the first fastener through the first hole and into the proximal and distal bone fragments and locking the head of the first fastener relative to the first hole, wherein the rotational travel of the head of the first fastener through the first hole is sufficient to cause a limited amount of compression of the distal fragment against the proximal fragment.
